(19) United States
(12) Patent Application Publication
(10) Pub. No.: US 2024/0041694 A1
(43) Pub. Date: Feb. 8, 2024

(54) THERMOTHERAPY DEVICE

(71) Applicant: CERAGEM Co., Ltd., Cheonan-si, Chungcheongnam-do (KR)

(72) Inventors: Dong Myoung Lee, Asan-si, Chungcheongnam-do (KR); Keun Young PAEK, Cheonan-si, Chungcheongnam-do (KR); Sang Ho Choi, Cheonan-si, Chungcheongnam-do (KR)

(21) Appl. No.: 18/264,410

(22) PCT Filed: Feb. 4, 2022
(86) PCT No.: PCT/KR2022/001767

§ 371 Date: Aug. 4, 2023

(30) Foreign Application Priority Data
Feb. 5, 2021 (KR) 10-2021-0016753

(51) Int. Cl. A61H 15/02; A61H 15/00

(52) U.S. Cl. CPC A61H 15/02; A61H 15/0078; A61H 2201/0207; A61H 2201/0119; A61H 2201/1669

(57) ABSTRACT

The present invention relates to a thermo-therapeutic apparatus. More particularly, the present invention relates to a thermo-therapeutic apparatus in which a heating unit for heating a ceramic unit can be supplied with a current from a power supply unit even in a state where the heating unit rotates together with the ceramic unit. To this end, the thermo-therapeutic apparatus comprises: a ceramic unit having an inner space formed therein; a heating unit having a heating element which is inserted in the inner space to directly heat the ceramic unit; a power supply unit supplying a current to the heating unit; and a support supporting the ceramic unit, wherein the heating unit can rotate relative to the power supply unit so as to allow the heating unit to rotate together with the ceramic unit.

### TECHNICAL FIELD

The present invention relates to a thermo-therapeutic apparatus. More particularly, the present invention relates to a thermo-therapeutic apparatus in which a heating unit for heating a ceramic unit can be supplied with a current from a power supply unit even in a state where the heating unit rotates together with the ceramic unit.

### BACKGROUND ART

Conventionally, in order to ease acute or chronic pain occurring in muscles and nervous tissues of a spine region due to continuous work in an improper posture for a long time or habituation of such a posture for a long time, to improve blood circulation in the body, or to relieve momentary muscle stiffness, a thermo-therapeutic apparatus which moves along body parts and improves blood circulation by stimulating a pain-producing part with heat has been widely used.

A conventional thermo-therapeutic apparatus used for such thermotherapy performs massage while a thermal ceramic moves along a user's body in a longitudinal direction, wherein the thermal ceramic is configured to massage the user's body while rotating in the process of moving back and forth repeatedly throughout the entire moving section. This is to allow the thermal ceramic to rotate naturally due to friction with a cover, because when the thermal ceramic does not rotate, friction between the thermal ceramic and the cover is maximized, and the cover may be quickly worn out.

Conventionally, in order to heat the rotating thermal ceramic, a non-rotating heating element connected to a power source is inserted into the thermal ceramic, but the thermal ceramic is configured to be spaced apart from the heating element so that the rotating thermal ceramic can rotate relative to the non-rotating heating element.

However, as the thermal ceramic and the heating element were spaced apart from each other, there was a problem in that the heat generated from the heating element was not smoothly transferred, thereby reducing the thermal therapy effect.

Accordingly, there is a need for improvement on this problem.
(Patent Document 1) Korean Patent Laid-Open Publication No. 2002-0039608 (published on May 27, 2002).

### DISCLOSURE

### Technical Tasks

The present invention has been made to solve the problems of the prior art described above, and the technical problem to be solved is to provide a thermo-therapeutic apparatus in which a heating unit for heating a ceramic unit can be supplied with a current from a power supply unit even in a state where the heating unit rotates together with the ceramic unit.

However, the technical problems to be solved by the present invention are not limited thereto, and other technical problems not mentioned will be clearly understood by those skilled in the art from the following description.

### Technical Solution

In order to solve the above technical problem, a thermo-therapeutic apparatus according to the present invention comprises: a ceramic unit having an inner space formed therein; a heating unit having a heating element which is inserted in the inner space to directly heat the ceramic unit; a power supply unit supplying a current to the heating unit; and a support supporting the ceramic unit, wherein the heating unit can rotate relative to the power supply unit so as to allow the heating unit to rotate together with the ceramic unit.

In this case, a heating surface in thermal contact with an inner circumferential surface of the ceramic unit may be provided around the heating element.

The power supply unit may include an electrode member for supplying an electric current, and both sides of the heating element in an axial direction may be provided with a conductive surface in electrical contact with the electrode member.

The electrode member may include a first electrode member disposed on one side in an axial direction of the heating element and a second electrode member disposed on the other side in the axial direction of the heating element.

The power supply unit may include a transmission member for transmitting a current supplied through the electrode member to the heating element.

The transmission member may rotate relative to the electrode member so that the transmission member rotates together with the heating element.

A contact surface in contact with an inner circumferential surface of the ceramic unit may be formed around the transmission member.

An insulating member for preventing the supplied current from moving to the ceramic unit may be provided on a radially outer side of the contact surface.

An insertion groove into which the electrode member is inserted and disposed may be formed in the transmission member.

The electrode member may include an electrode terminal through which the supplied current moves, and an electrode holder for fixing a position of the electrode terminal.

The electrode terminal may include an electrode head in electrical contact with an inner circumferential surface of the insertion groove, and an electrode body elastically deformed so that the electrode head presses the inner circumferential surface of the insertion groove.

A support groove into which the electrode head is inserted and supported may be formed on the inner circumferential surface of the insertion groove.

The heating unit may be provided with an elastic deformation member for pressing the inner circumferential surface of the ceramic unit.

### Advantageous Effects

The thermo-therapeutic apparatus of the present invention having the above configuration is configured so that the heating unit for heating the ceramic unit rotates together with the ceramic unit. Accordingly, as the ceramic unit and the heating unit are placed in direct contact with each other, the heat generated from the heating unit is smoothly transferred to the ceramic unit, thereby enhancing the thermal therapy effect.

In addition, as the heat is directly transferred from the heating unit to the ceramic unit, heat loss is minimized, thereby improving the power consumption efficiency of the thermo-therapeutic apparatus.

Further, since the current is stably supplied even in a state in which the heating unit rotating together with the ceramic unit rotates relative to the power supply part, it is possible to secure the operation stability of the thermo-therapeutic apparatus.

It should be understood that the effects of the present invention are not limited to the above effects, and include all effects that can be inferred from the configuration of the invention described in the detailed description or claims of the present invention.

### DESCRIPTION OF DRAWINGS

FIG. 1 is a cross-sectional view showing a thermo-therapeutic apparatus according to an embodiment of the present invention.

FIG. 2 is a cross-sectional view illustrating a state in which a ceramic unit and a heating unit are coupled according to an embodiment of the present invention.

FIG. 3 is a cross-sectional view illustrating an exploded state of a ceramic unit and a heating unit according to an embodiment of the present invention.

FIG. 4 is a cross-sectional view illustrating a state in which a ceramic unit and a heating unit are coupled according to another embodiment of the present invention.

FIG. 5 is a cross-sectional view illustrating a state in which a ceramic unit and a heating unit are coupled according to still another embodiment of the present invention.

FIG. 6 is a cross-sectional view illustrating a state in which a ceramic unit and a heating unit are coupled according to yet another embodiment of the present invention.

FIGS. 7 and 8 are cross-sectional views illustrating a heating unit and a power supply unit according to another embodiment of the present invention.

FIG. 9 is a side view illustrating a heating unit according to another embodiment of the present invention.

### EMBODIMENTS OF THE INVENTION

Hereinafter, with reference to the accompanying drawings, embodiments of the present invention will be described in detail so as to be easily implemented by one of ordinary skill in the art to which the present invention pertains. The present invention may be embodied in a variety of forms and is not limited to the embodiments described herein. In order to clearly describe the present invention in the drawing, parts irrelevant to the description are omitted from the drawings; and throughout the specification, same or similar components are referred to as like reference numerals.

In the specification, terms such as “comprise” or “have” are intended to designate that a feature, number, step, operation, component, part or combination thereof described in the specification is present, but should not be construed to preclude the possibility of the presence or addition of one or more other features, numbers, steps, operations, components, parts or combinations thereof. In addition, when a part such as a layer, film, region, plate, etc. is said to be “on” another part, this includes not only the case where the part is “directly on” the another part, but also the case where there is still another part between them. Conversely, when a part such as a layer, film, region, plate, etc. is said to be “under” another part, this includes not only the case where the part is “directly under” the another part, but also the case where there is still another part between them.

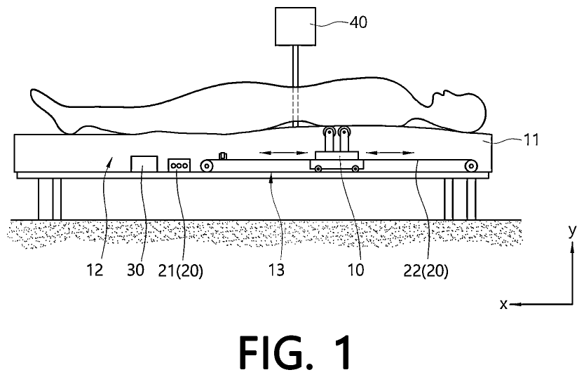

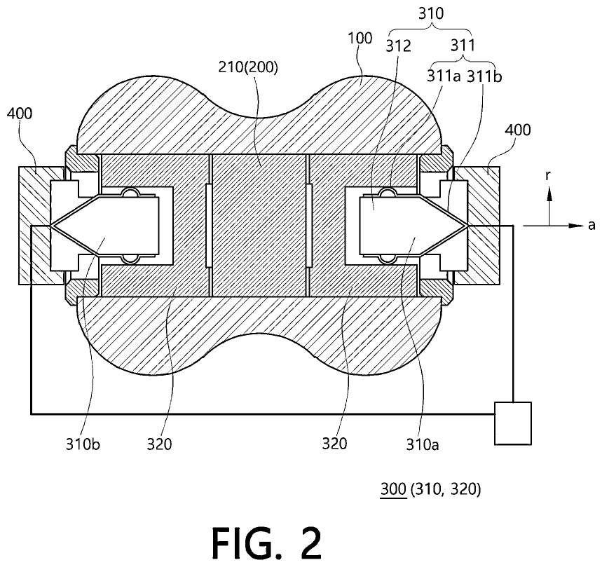

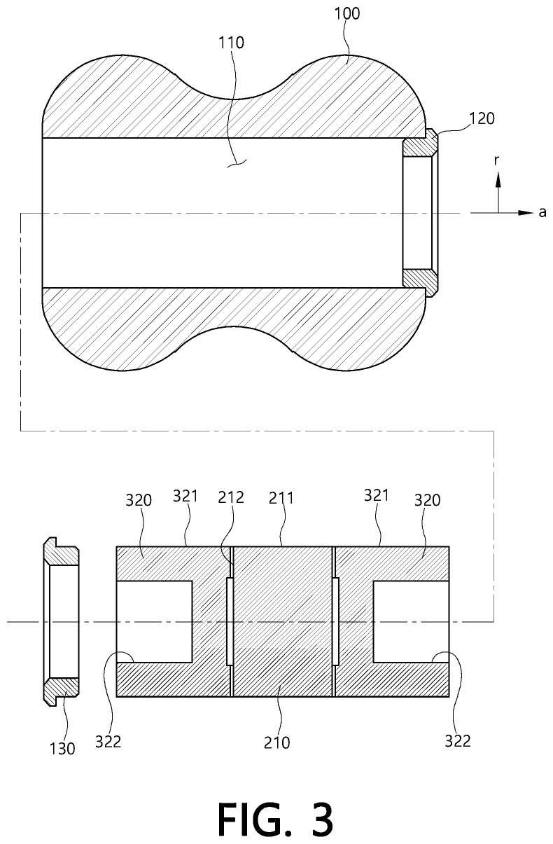

FIG. 1 is a cross-sectional view showing a thermo-therapeutic apparatus according to an embodiment of the present invention, FIG. 2 is a cross-sectional view illustrating a state in which a ceramic unit and a heating unit are coupled according to an embodiment of the present invention, and FIG. 3 is a cross-sectional view illustrating an exploded state of a ceramic unit and a heating unit according to an embodiment of the present invention.

As shown in FIG. 1, a thermo-therapeutic apparatus according to an embodiment of the present invention includes a ceramic module 10, a driving unit 20 for moving the ceramic module 10, a control unit 30 for controlling an operation of the driving unit 20, and an input unit 40 for inputting a thermal therapy pattern desired by a user.

In this case, the thermo-therapeutic apparatus may include a main mat 11 used for the user's upper body and its spine, and an auxiliary mat 12 used for the user's lower body. In addition, it may include a mounting unit 13 for placing and supporting the main mat 11 and the auxiliary mat 12, if necessary.

The ceramic module 10 can massage the spine while moving in a longitudinal direction (x) along the user's spine. This ceramic module 10 can provide the user with a thermal compress and massage effect by using high-temperature heat generated by a current supplied from the power supply unit 300 to be described later.

The ceramic module 10 may also be configured to provide the user with the thermal compress and massage effect by using not only high-temperature heat but also far-infrared rays.

The ceramic unit 100 provided in the ceramic module 10 may be formed in a roller type, but is not limited thereto, and may have various shapes and structures as long as the ceramic unit 100 is configured to rotate while the ceramic module 10 is moved. In addition, when the ceramic unit 100 is formed of a material such as ceramic, far-infrared rays are generated during the use of the thermo-therapeutic apparatus, which can improve the thermal therapy effect. However, it is not necessarily limited to this material, and may be formed of any other materials as long as they can transfer heat to the user's body and provide a thermal therapy effect.

As shown in FIG. 2, the ceramic module 10 includes a ceramic unit 100 having an inner space 110 formed therein, a heating unit 200 having a heating element 210 inserted in the inner space 110 to directly heat the ceramic unit 100, a power supply unit 300 supplying a current to the heating unit 200, and a support 400 supporting the ceramic unit 100.

Here, a PTC heater may be used as the heating element 210, but the present invention is not necessarily limited thereto, and a lamp or any various heating means capable of being heated by supplying current may be used.

In addition, the driving unit 20 may include a first driving member for moving the ceramic module 10 in the user's longitudinal direction (x). The first driving member may include a driving motor 21 for reciprocating the ceramic module 10, and a conveying member 22.

The driving motor 21 rotates by receiving current, and the conveying member 22 is connected to the driving motor 21 and receives the rotational force according to the rotation of the driving motor 21 to move the ceramic module 10.

The conveying member 22 is connected to the ceramic module 10, and is used to convey the ceramic module 10 in one or the other direction along the user's longitudinal direction (x) according to forward or reverse rotation of the driving motor 21.

The conveying member 22 may be selected from a conveying belt, a conveying chain, and a conveying rope, but is not limited thereto, and any various means for conveying the object using the driving force of the driving motor 21 may be used, such as a lead screw, or a rack and pinion.

The driving motor 21 may also be configured to provide a driving force while being spaced apart from the ceramic module 10 or to provide a driving force while being inserted into the ceramic module 10.

In addition, the driving unit 20 may include a second driving member for increasing the vertical height of the ceramic module 10 so that a pressing force is applied to the user or lowering the vertical height of the ceramic module 10 so that the pressing force is removed.

As shown in FIG. 2, since the heating unit 200 is configured to rotate together with the ceramic unit 100 during the thermal therapy process, the ceramic unit 100 and the heating unit 200 are arranged in direct contact with each other. Accordingly, the heat generated from the heating unit 200 is directly transferred to the ceramic unit 100, thereby improving the thermal therapy effect.

Such a heating unit 200 may be configured to evenly and directly contact the entire inner circumferential surface of the inner space 110 formed in the ceramic unit 100, but is not necessarily limited thereto, and can also be configured to directly contact only a certain portion as long as the heat generated from the heating unit 200 can be smoothly transferred to the ceramic unit 100.

As such, since the heat is directly transferred from the heating unit 200 to the ceramic unit 100, heat loss is minimized, thereby improving the power consumption efficiency of the thermo-therapeutic apparatus. Although the power supply unit 300 is fixed in a non-rotating state, the heating unit 200 is stably supplied with current through the power supply unit 300 while rotating relative to the power supply unit 300, thereby enabling stable operation of the thermo-therapeutic apparatus.

The power supply unit 300 may be provided with an electrode member 310 to be described later, wherein the electrode member 310 may be configured to smoothly supply current even when the heating unit 200 rotates together with the ceramic unit 100.

As shown in FIG. 3, a first bushing 120 is provided on one side of the ceramic unit 100, and a second bushing 130 is provided on the other side of the ceramic unit 100 so that the ceramic unit 100 is rotatably supported by the support 400.

That is, the heating unit 200 is inserted into the inner space 110 of the ceramic unit 100 in a state where the first bushing 120 is coupled to one side of the ceramic unit 100. In this case, the heating unit 200 may be inserted in a state in which the heating element 210 and the transmission member 320 to be described later are assembled with each other, or may also be sequentially inserted in a state in which the heating element 210 and the transmission member 320 are separated. That is, the transmission member 320 disposed on one side of the ceramic unit 100 is first inserted, the heating element 210 is inserted, and then the transmission member 320 disposed on the other side of the ceramic unit 100 is inserted.

As shown in FIG. 3, a heating surface 211 in thermal contact with the inner circumferential surface of the ceramic unit 100 may be provided around the heating element 210. That is, since the heating surface 211 comes into direct thermal contact with the inner circumferential surface of the ceramic unit 100, the heat can be smoothly transferred to the ceramic unit 100.

As shown in FIG. 2, the power supply unit 300 may include an electrode member 310 for supplying current. The electrode member 310 functions as a current passage so that the current is supplied to the heating element 210. In addition, a conductive surface 212 in electrical contact with the electrode member 310 may be provided on both sides in an axial direction (a) of the heating element 210. The current supplied through the electrode member 310 moves to the heating element 210 through the conductive surface 212.

The electrode member 310 may include a first electrode member 310a disposed on one side in an axial direction (a) of the heating element 210, and a second electrode member 310b disposed on the other side in the axial direction (a) of the heating element 210. As an example, the current supplied through the first electrode member 310a moves to the heating element 210 through the conductive surface 212 provided on one side of the heating element 210, and the current moved to the other side of the heating element 21 moves to the second electrode member 310b through the conductive surface 212 provided on the other side of the heating element 210.

As shown in FIGS. 2 and 3, the power supply unit 300 may include a transmission member 320 that transmits the current supplied through the electrode member 310 to the heating element 210. As described above, since the heating unit 200 is configured to rotate together with the ceramic unit 100, the heating element 210 continuously rotates in the process of using the thermo-therapeutic apparatus. In this case, the electrode member 310 may be configured to be in direct electrical contact with the heating element 210, but as described above, the transmission member 320 for transmitting current may be provided between the electrode member 310 and the heating element 210 so that the current supplied through the electrode member 310 passes to the heating element 210 through the transmission member 320. In addition, the transmission member 320 may be configured to rotate together with the heating element 210 without rotating relative to the heating element 210. With this configuration, since there is no relative rotation between the heating element 210 and the transmission member 320, the heating element 210 can be prevented from being worn out. The heat generated from the heating element 210 may also be configured to be transferred to the ceramic unit 100 through the transmission member 320. To this end, a contact surface 321 in contact with the inner circumferential surface of the ceramic unit 100 may be formed around the transmission member 320 as described later. The transmission member 320 is preferably formed of a material capable of moving current and heat. For example, when the transmission member 320 is formed of a material having high heat transfer efficiency, such as aluminum, the heat moves smoothly at the same time as the current flows, so that the ceramic unit 100 can be efficiently heated.

As shown in FIG. 3, the transmission member 320 may rotate relative to the electrode member 310 so that the transmission member 320 rotates together with the heating element 210. As described above, when the transmission member 320 rotates together with the heating element 210, the heating element 210 can be prevented from being worn out. As such, the transmission member 320 is configured to rotate relative to the electrode member 310 so that current can be stably supplied through the electrode member 310 even when the transmission member 320 rotates. As such, as the transmission member 320 and the electrode member 310 rotate relative to each other, wear may occur between them. However, since the cost of replacing the transmission member 320 or the electrode member 310 is relatively low compared to the cost of replacing the heating element 210, maintenance and repair costs can be reduced with this configuration. In addition, if a component having a relatively low replacement cost among the transmission member 320 and the electrode member 310 is configured to be easily worn out, maintenance and repair costs can be further reduced.

As shown in FIG. 3, a contact surface 321 in contact with the inner circumferential surface of the ceramic unit 100 may be formed around the transmission member 320. When such a contact surface 321 is formed on the transmission member 320, the ceramic unit 100 rotates together with the heating element 210, and thus, abrasion does not occur between them.

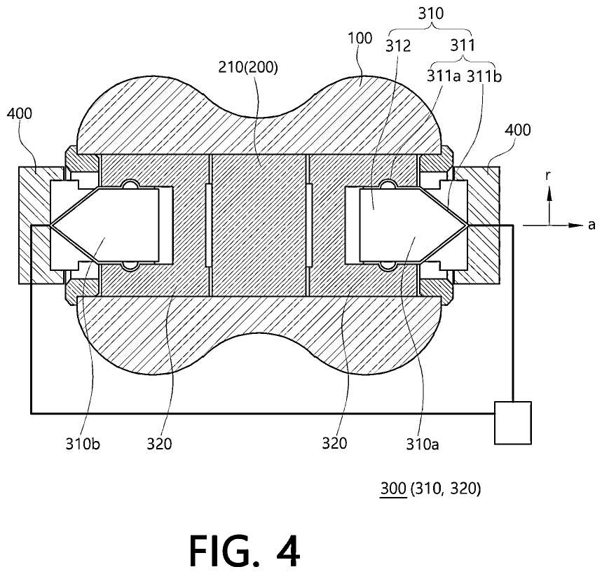

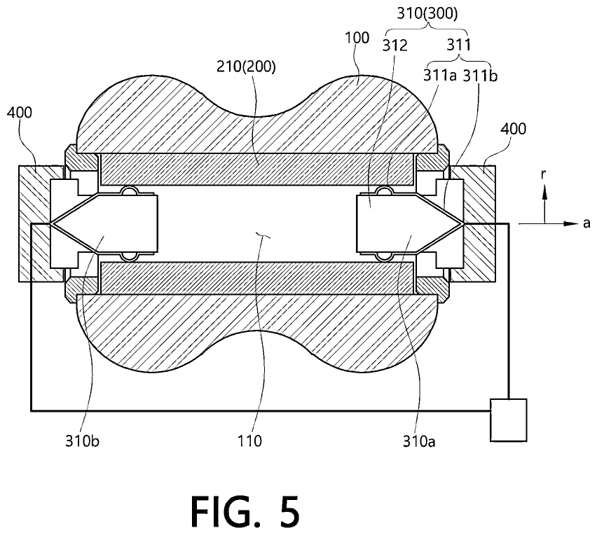

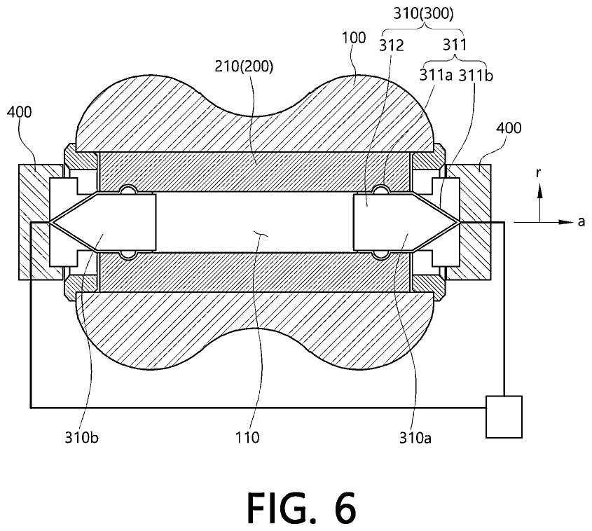

FIG. 4 is a cross-sectional view illustrating a state in which a ceramic unit and a heating unit are coupled according to another embodiment of the present invention, FIG. 5 is a cross-sectional view illustrating a state in which a ceramic unit and a heating unit are coupled according to still another embodiment of the present invention, and FIG. 6 is a cross-sectional view illustrating a state in which a ceramic unit and a heating unit are coupled according to yet another embodiment of the present invention.

As shown in FIG. 4, in the case of the ceramic module 10 according to another embodiment of the present invention, it includes a ceramic unit 100 having an inner space 110 formed therein, a heating unit 200 having a heating element 210 inserted in the inner space 110 to directly heat the ceramic unit 100, a power supply unit 300 supplying a current to the heating unit 200, and a support 400 supporting the ceramic unit 100, wherein the heating unit 200 is configured to rotate together with the ceramic unit 100 during the thermal therapy process, so the ceramic unit 100 and the heating unit 200 are arranged in direct contact with each other, and therefore, the heat generated from the heating unit 200 is directly transferred to the ceramic unit 100, thereby improving the thermal therapy effect, which is the same as in the above embodiment. However, as will be described later, a support groove 322a into which a part of the electrode member 310 is inserted and disposed is formed in the transmission member 320 provided in the power supply unit 300, which is partially different from the above embodiment in terms of configuration.

In addition, as shown in FIGS. 5 and 6, in the case of the ceramic module 10 according to still another embodiment of the present invention, it includes a ceramic unit 100 having an inner space 110 formed therein, a heating unit 200 having a heating element 210 inserted in the inner space 110 to directly heat the ceramic unit 100, a power supply unit 300 supplying a current to the heating unit 200, and a support 400 supporting the ceramic unit 100, wherein the heating unit 200 is configured to rotate together with the ceramic unit 100 during the thermal therapy process, so the ceramic unit 100 and the heating unit 200 are arranged in direct contact with each other, and therefore, the heat generated from the heating unit 200 is directly transferred to the ceramic unit 100, thereby improving the thermal therapy effect, which is the same as in the above embodiment. In this case, an inner space may be formed inside the heating element 210, and the electrode member 310 provided in the power supply unit 300 may be disposed in this inner space. In addition, a heating surface 211 in direct thermal contact with the inner circumferential surface of the ceramic unit 100 may be formed outside the heating element 210. However, the configurations of the two are partially different in that in the case of FIG. 5, the electrode member 310 is supported so as to simply come into contact with the inner circumferential surface of the heating element 210, whereas in the case of FIG. 6, a support groove 322a in which a part of the electrode member 310 is inserted and disposed is formed in the inner circumferential surface of the heating element 210.

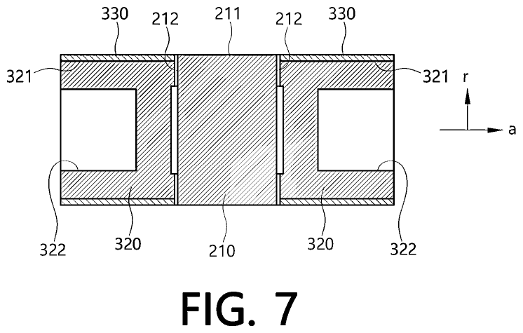

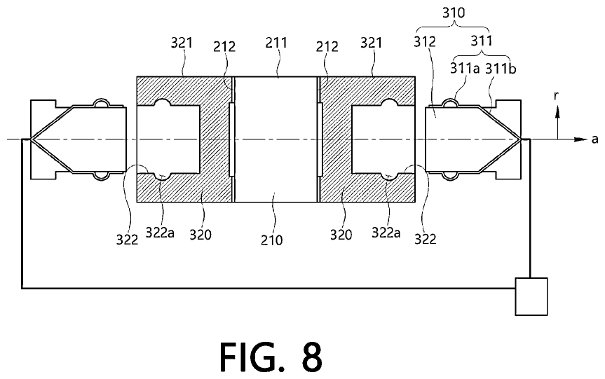

FIGS. 7 and 8 are cross-sectional views illustrating a heating unit and a power supply unit according to another embodiment of the present invention.

As shown in FIG. 7, an insulating member 330 for preventing the supplied current from moving to the ceramic unit 100 may be provided outside the contact surface 321 in a radial direction (r). As described above, the current supplied through the electrode member 310 moves to the heating element 210 via the transmission member 320. However, as described above, a contact surface 321 is formed around the transmission member 320 so that the transmission member 320 rotates together with the ceramic unit 100. However, when the ceramic unit 100 is made of a material through which current can flow, such as aluminum, the current supplied through the electrode member 310 moves through the contact surface 321 and the ceramic unit 100.

In this case, there may be a problem that the heating element 210 is not normally heated. Therefore, as described above, the insulating member 330 is provided outside the contact surface 321 so that the supplied current does not move to the ceramic unit 100 but can move only through the heating element 210, whereby the heating element 210 can be heated normally.

As shown in FIG. 8, an insertion groove 322 into which the electrode member 310 is inserted and disposed may be formed in the transmission member 320. When the insertion groove 322 is formed in this way, the electrode member 310 can be installed after accurately confirming the position where it is inserted. In addition, the operator can simply install the electrode member 310 by inserting it into the insertion groove 322, thereby improving workability.

In addition, the electrode member 310 may include an electrode terminal 311 through which the supplied current moves, and an electrode holder 312 for fixing the position of the electrode terminal 311. That is, when the electrode member 310 is prepared in a state in which the electrode terminal 311 is fixed to the outer circumferential surface of the electrode holder 312, an operator can simply install it by holding the electrode holder 312 and inserting the same into the insertion groove 322 of the transmission member 320 described above.

Here, as shown in FIG. 8, the electrode terminal 311 may include an electrode head 311a in electrical contact with the inner circumferential surface of the insertion groove 322, and an electrode body 311b elastically deformed so that the electrode head 311a presses the inner circumferential surface of the insertion groove 322. That is, while the electrode terminal 311 is inserted into the insertion groove 322 by a worker, the electrode body 311b to which the electrode head 311a is connected is elastically deformed. After the electrode terminal 311 is inserted into the insertion groove 322, the electrode body 311b is elastically restored to press the inner circumferential surface of the insertion groove 322, thereby enabling electrically stable contact. In addition, as described above, even if the electrode terminal 311 is partially worn while the transmission member 320 rotates together with the ceramic unit 100, the electrode body 311b is elastically restored to continuously press the electrode head 311a to electrically contact the inner circumferential surface of the insertion groove 322, thereby enabling an electrically stable connection.

In addition, a support groove 322a into which the electrode head 311a is inserted and supported may be formed on the inner circumferential surface of the insertion groove 322. When the support groove 322a is formed as described above, the electrode body 311b is elastically deformed in the process of installing the electrode terminal 311 and then elastically restored to insert and support the electrode head 311a into the support groove 322a, whereby the electrode head 311a continuously presses the support groove 322a, thereby preventing the electrode terminal 311 from being arbitrarily separated during use.

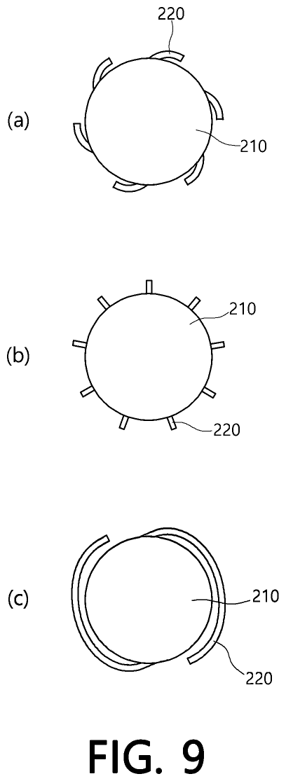

FIG. 9 is a side view illustrating a heating unit according to another embodiment of the present invention.

As shown in FIG. 9, the heating unit 200 may include an elastic deformation member 220 that presses the inner circumferential surface of the ceramic unit 100. Basically, the elastic deformation member 220 is elastically deformed in the process of assembling the heating unit 200 to the ceramic unit 100, and is elastically restored after assembly to press the inner circumferential surface of the ceramic unit 100. Therefore, the heat generated from the heating unit 200 can be directly transferred to the ceramic unit 100 through the elastic deformation member 220 in a conductive manner, thereby improving the thermal therapy effect.

In this case, not only the heating unit 200 but also the ceramic unit 100 undergoes thermal expansion due to the heat generated through the heating unit 200 in the process of using the thermo-therapeutic apparatus. If the materials of the heating unit 200 and the ceramic unit 100 are different from each other, the degrees of thermal expansion will be different. For example, when the ceramic unit 100 is formed of a ceramic material and the heating unit 200 is formed of an aluminum material, the degree of thermal expansion of the heating unit 200 is greater than that of the ceramic unit 100. Therefore, a phenomenon in which the heating unit 200 presses the inner circumferential surface of the ceramic unit 100 occurs during use of the thermo-therapeutic apparatus, which may cause damage to the ceramic unit 100. As described above, when the elastic deformation member 220 is provided in the heating unit 200, the elastic deformation member 220 is elastically deformed during the thermal expansion of the heating unit 200. Thus, the force for pressing the inner circumferential surface of the ceramic unit 100 can be reduced, thereby effectively preventing the ceramic unit 100 from being damaged.

At least one elastic deformation member 220 may be provided along the circumference of the heating unit 200. As shown in (a) of FIG. 9, the elastic deformation member 220 is preferably configured so that the front end of the elastic deformation member 220 is disposed adjacent to the outer circumferential surface of the heating unit 200, but is spaced apart from the outer circumferential surface of the heating unit 200 by a certain distance so as to be elastically deformed. With this configuration, when thermal expansion occurs in the heating unit 200, the elastic deformation member 220 is elastically deformed so that the separation distance between the front end of the elastic deformation member 220 and the outer circumferential surface of the heating unit 200 is reduced, thereby reducing the force pressing the inner circumferential surface of the ceramic unit 100. Alternatively, as shown in (b) of FIG. 9, the elastic deformation member 220 may also be configured to extend in a radial direction. With this configuration, when thermal expansion occurs in the heating unit 200, the elastic deformation member 220 is elastically deformed in a bending manner, thereby reducing the force pressing the inner circumferential surface of the ceramic unit 100. Further, as shown in (c) of FIG. 9, the elastic deformation member 220 may also be configured to partially cover the outer circumferential surface of the heating unit 200. That is, in (a) and (c) of FIG. 9, the basic operations in which the elastic deformation member 220 is elastically deformed when the heating unit 200 thermally expands are similar to each other, but the elastic deformation member 220 shown in (c) of FIG. 9 is formed to be longer than that shown in (a) of FIG. 9. With this configuration, the contact area between the elastic deformation member 220 and the inner circumferential surface of the ceramic unit 100 can be increased, thereby improving the heat transfer effect.

Although an embodiment of the present invention have been described above, the spirit of the present invention is not limited to the embodiment presented in the subject specification; and those skilled in the art who understands the spirit of the present invention will be able to easily suggest other embodiments through addition, changes, elimination, and the like of elements without departing from the scope of the same spirit, and such other embodiments will also fall within the scope of the present invention.

1. A thermo-therapeutic apparatus comprising:
a ceramic unit having an inner space formed therein;
a heating unit having a heating element which is inserted in the inner space to directly heat the ceramic unit;
a power supply unit supplying a current to the heating unit; and
a support supporting the ceramic unit,
wherein the heating unit rotates relative to the power supply unit so as to allow the heating unit to rotate together with the ceramic unit.

2. The thermo-therapeutic apparatus according to claim 1, wherein a heating surface in thermal contact with an inner circumferential surface of the ceramic unit is provided around the heating element.

3. The thermo-therapeutic apparatus according to claim 1, wherein the power supply unit includes an electrode member for supplying an electric current, and
wherein both sides of the heating element in an axial direction is provided with a conductive surface in electrical contact with the electrode member.

4. The thermo-therapeutic apparatus according to claim 3, wherein the electrode member includes a first electrode member disposed on one side in an axial direction of the heating element and a second electrode member disposed on the other side in the axial direction of the heating element.

5. The thermo-therapeutic apparatus according to claim 3, wherein the power supply unit includes a transmission member for transmitting a current supplied through the electrode member to the heating element.

6. The thermo-therapeutic apparatus according to claim 5, wherein the transmission member rotates relative to the electrode member so that the transmission member rotates together with the heating element.

7. The thermo-therapeutic apparatus according to claim 6, wherein a contact surface in contact with an inner circumferential surface of the ceramic unit is formed around the transmission member.

8. The thermo-therapeutic apparatus according to claim 7, wherein an insulating member for preventing the supplied current from moving to the ceramic unit is provided on a radially outer side of the contact surface.

9. The thermo-therapeutic apparatus according to claim 6, wherein an insertion groove into which the electrode member is inserted and disposed is formed in the transmission member.

10. The thermo-therapeutic apparatus according to claim 9, wherein the electrode member includes an electrode terminal through which the supplied current moves, and an electrode holder for fixing a position of the electrode terminal.

11. The thermo-therapeutic apparatus according to claim 10, wherein the electrode terminal includes an electrode head in electrical contact with an inner circumferential surface of the insertion groove, and an electrode body elastically deformed so that the electrode head presses the inner circumferential surface of the insertion groove.

12. The thermo-therapeutic apparatus according to claim 11, wherein a support groove into which the electrode head is inserted and supported is formed on the inner circumferential surface of the insertion groove.

13. The thermo-therapeutic apparatus according to claim 1, wherein the heating unit is provided with an elastic deformation member for pressing the inner circumferential surface of the ceramic unit.
